(19) United States
(12) Patent Application Publication
(10) Pub. No.: US 2016/0096885 A1
(43) Pub. Date: Apr. 7, 2016

(54) DISTINCT EFFECTS OF IFN-GAMMA AND IL-17 ON TL1A MODULATED INFLAMMATION AND FIBROSIS

(71) Applicant: CEDARS-SINAL MEDICAL CENTER, Los Angeles, CA (US)

(72) Inventors: David Q. Shih, La Crescenta, CA (US); Stephan R. Targan, Santa Monica, CA (US); Janine Bilsborough, Simi Valley, CA (US)

(73) Assignee: Cedars-Sinal Medical Center, Los Angeles, CA (US)

(21) Appl. No.: 14/890,699

(22) PCT Filed: May 16, 2014
(86) PCT No.: PCT/US2014/038333

§ 371 Date: Nov. 12, 2015

Related U.S. Application Data
(60) Provisional application No. 61/824,891, filed on May 17, 2013.

(51) Int. Cl. C07K 16/24; C12Q 1/68; G01N 33/68; A61K 39/395

(52) U.S. Cl. CPC C07K 16/244; A61K 39/3955; C12Q 1/6883; G01N 33/6893; A61K 2039/505

(57) ABSTRACT

Described herein are methods and compositions related to Inflammatory Bowel Disease. Specifically TL1A drives regional intestinal inflammation and fibrosis and is differentially modulated by IFN gamma and IL-17a. In one embodiment, the present invention is a method of diagnosing a condition in a subject by determining the presence or absence of IFN gamma and/or IL-17 and diagnosing the subject.

### FIELD OF THE INVENTION

The claimed invention relates to prognosis, diagnosis and treatment of inflammatory bowel disease and related conditions, including methods and compositions for medical therapies.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Under chronic injury, inflammation of the intestine occurs. Most of the time, there is physiological healing leading to normal homeostasis. In pathological healing, gut fibrosis may occur, leading to intestinal fibrosis and strictures. Inflammatory bowel disease (IBD) such as Crohn's disease (CD) are chronic inflammatory conditions with pathological features such as patchy transmural inflammation and fibrostenosis.

Previous studies show that TL1A may drive intestinal inflammation through enhancing Th1, Th2 and Th17 effector function, TL1A appears to also drive fibrogenesis through increased number of fibroblasts and activated fibroblasts. SNRs of TL1A (TNFSF15), a TNF superfamily member, have been found to be associated with IBD, and certain TNFSF15 haplotypes have been found to be associated with increased TL1A expression and have a higher risk of small bowel surgery. Constitutive TL1A expression in mice has been found to confer worsened murine ileo-cecal inflammation, and intestinal fibrostenosis.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts background information on TL1A.

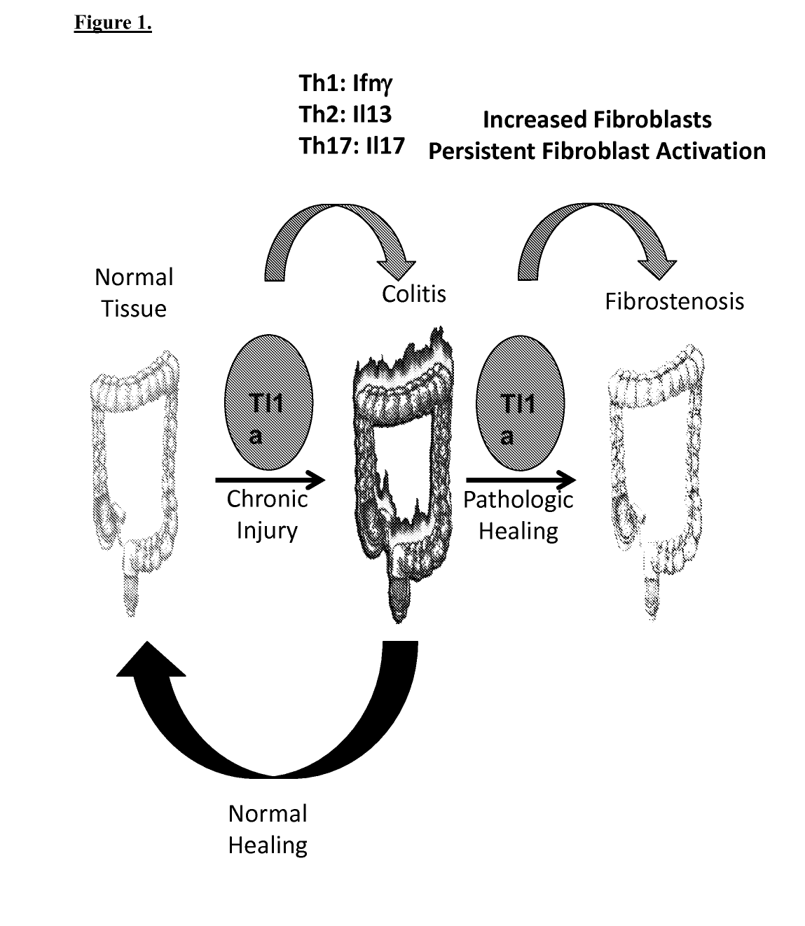

FIG. 2 depicts, in accordance with an embodiment herein, an experimental outline of the inventors.

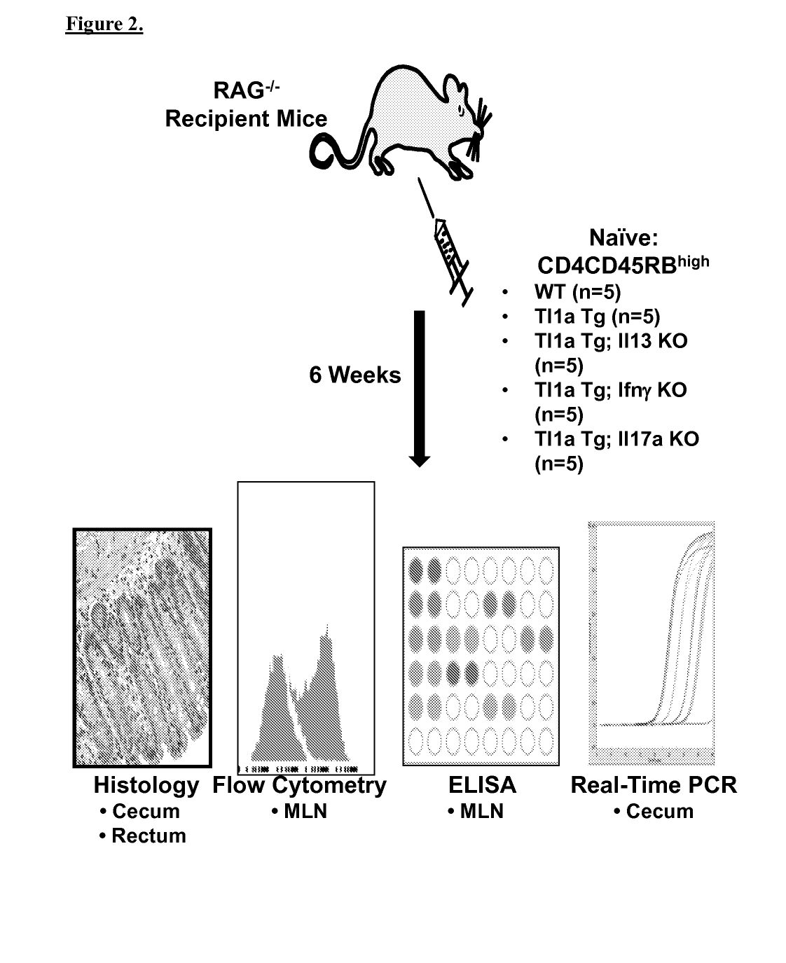

FIG. 3 depicts, in accordance with an embodiment herein, gross colonic inflammation is modulated by Th effector response. Gross Colonic inflammation is represented by increased erythema and swelling. WT mice in the adoptive transfer model have increased inflammation in the rectum, in contrast to WT, the inflammation was shifted to the cecum under T11a driven condition. Combining effector cytokine deficiency with sustained TL1A expression modulated regional gross inflammation. Under TL1a driven condition, IFNg deficiency led to pan colitis, IL13 deficiency shifted the inflammation to the WT pattern, and IL17 deficiency reduced overall colonic inflammation.

FIG. 4 depicts, in accordance with an embodiment herein, IL17a KO reduced TL1A associated proximal colitis. Compared to TL1A tg alone, there is no differences in inflammation with IL13 and IFNg deficiency. Shown here are results indicating that IL17a deficiency significantly reduced the severity of TL1A associated cecal inflammation.

FIG. 5 depicts, in accordance with an embodiment herein, rectal sparing of inflammation is modulated by Th effector response. Shown here are results indicating that IFNg deficiency significantly abrogated the severity of TL1A associated sparing of rectal inflammation. Additionally, IL17a deficiency further improved the rectal sparing associated with sustained TL1A expression.

FIG. 6 depicts, in accordance with an embodiment herein, IFN gamma is reduced with IL17a deficiency under TL1A driven condition Consistent with previous findings, sustained expression of TL1A led to increased percentage of CD4+IFNg+ cells and decreased percentage of CD4+I117a+ cells as compared to WT. Under TL1A driven condition, IL17a deficiency reduced CD4+IFNg+ cells.

FIG. 7 depicts, in accordance with an embodiment herein, sustained TL1A expression with IFN gamma and IL17a deficiency increased IL17f, I117a production was increased in mice with sustained TL1A expression. However, IFNg and I113 deficiency under TL1A driven condition didn't alter I117a production when compared to TL1A tg mice alone. Another major IL17 cytokine is IL17F. The results further suggest that I117f production was increased in RAG mice that received T11a Tg, naïve T cells with deficiencies in IFNg and IL17 KO.

FIG. 8 depicts, in accordance with an embodiment herein, IFN gamma deficiencies modulate TL1A driven TH-2 responses. Th2 associated cytokines production were also measured. I14 and I113 were increased in both IFNg deficiency and I117a deficiency under TL1A driven condition. The enhancement in TH2 related cytokine is higher with IFNg KO than IL17a. KO.

FIG. 9 depicts, in accordance with an embodiment herein, increased IL10 under TL driven condition with IL17a deficiency. IL10, a regulatory cytokine, was also measured and found to be significantly increased in RAG mice that received T11a Tg, IL17 KO naïve T cells.

FIG. 10 depicts, in accordance with an embodiment herein, a summary of inflammation for IFN gamma and IL17a cytokines.

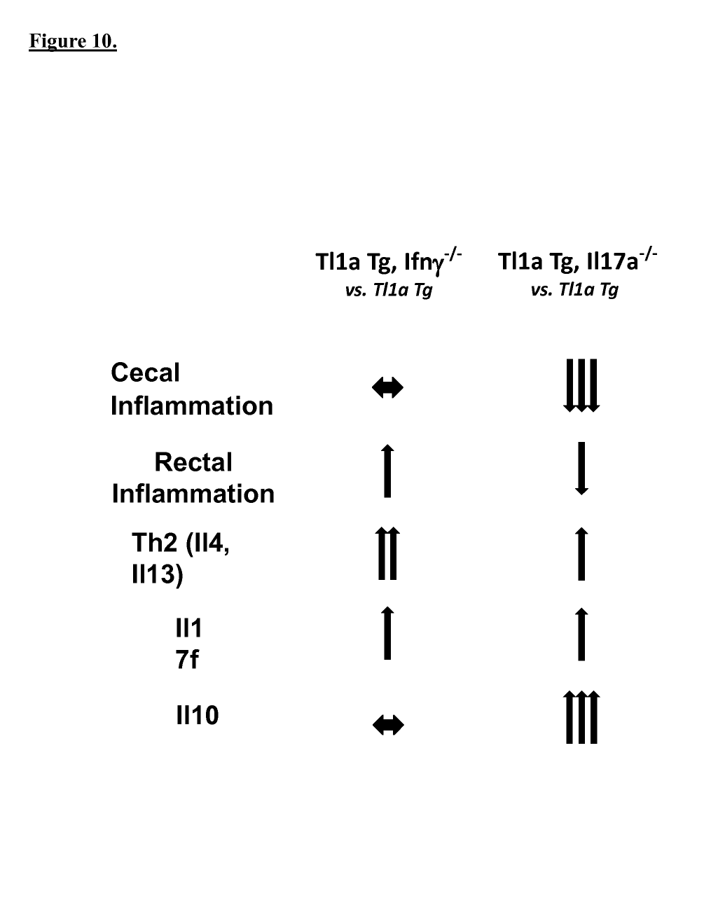

FIG. 11 depicts, in accordance with an embodiment herein, IL17a deficiency reduces TL1A mediated gut fibrosis. Under T11a driven condition, blocking IFNg has no effect on fibrosis whereas blocking IL17 significantly reduces collagen deposition when compared to TL1A transgenic mice.

FIG. 12 depicts, in accordance with an embodiment herein, activated fibroblasts are increased by IFN gamma KO but reduced by IL17a KOUnder TL1A driven condition, activated myofibroblasts were increased compared to WT, blocking IFNg further increased activated myofibroblasts whereas blocking IL17 reduced activated myofibroblasts when compared to TL1A transgenic mice.

FIG. 13 depicts, in accordance with an embodiment herein, under TL1A driven conditions, IL17a modulates fibrogenic factors expression. For different fibrosis patterns, it was discovered that under T11a driven condition, I117a deficiency reduced expression of pro-fibrotic factors including TGFb1, and also reduced expressions of fibrotic mediators Col1a2 and vimentin, which is consistent with reduced intestinal fibrosis in these mice.

FIG. 14 depicts, in accordance with an embodiment herein, a summary of fibrosis for IFN gamma and IL17a cytokines.

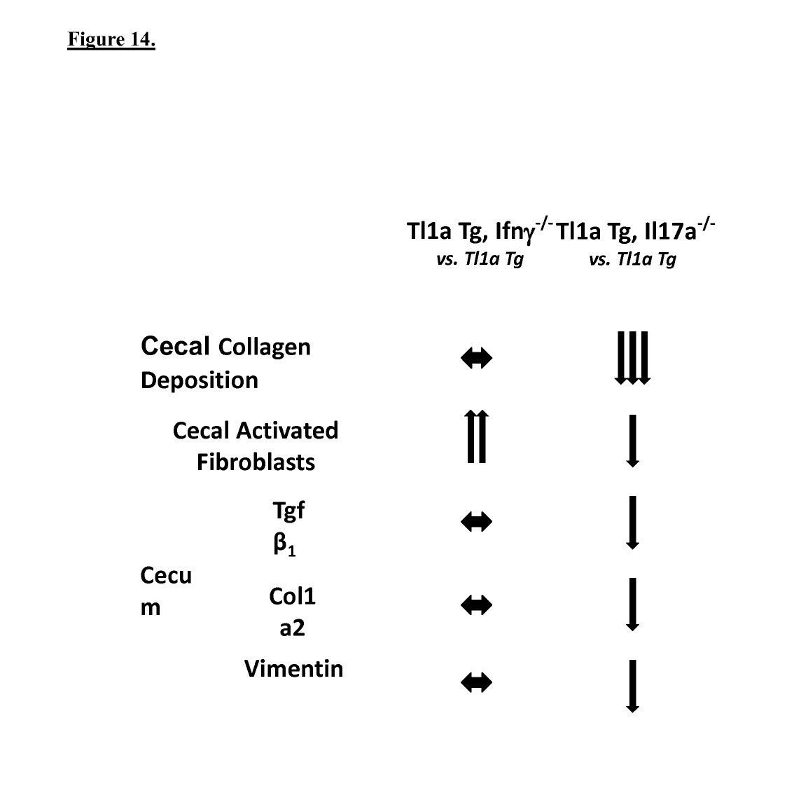

FIG. 15 depicts, in accordance with an embodiment herein, a summary of fibrosis for IL13.

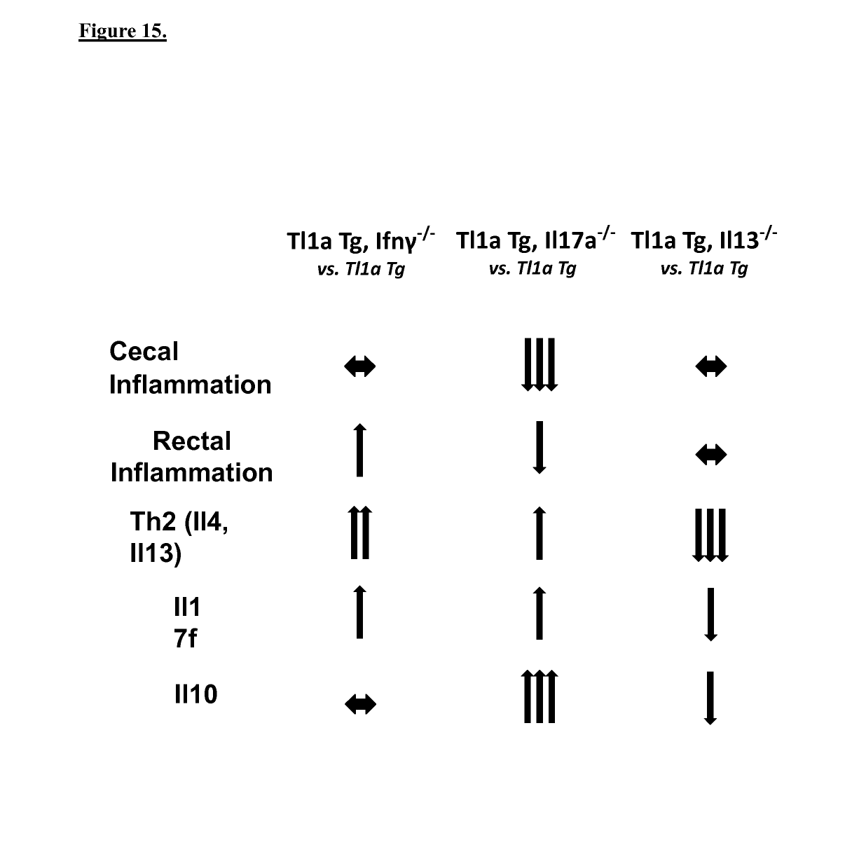

FIG. 16 depicts, in accordance with an embodiment herein, a summary of fibrosis for IL13.

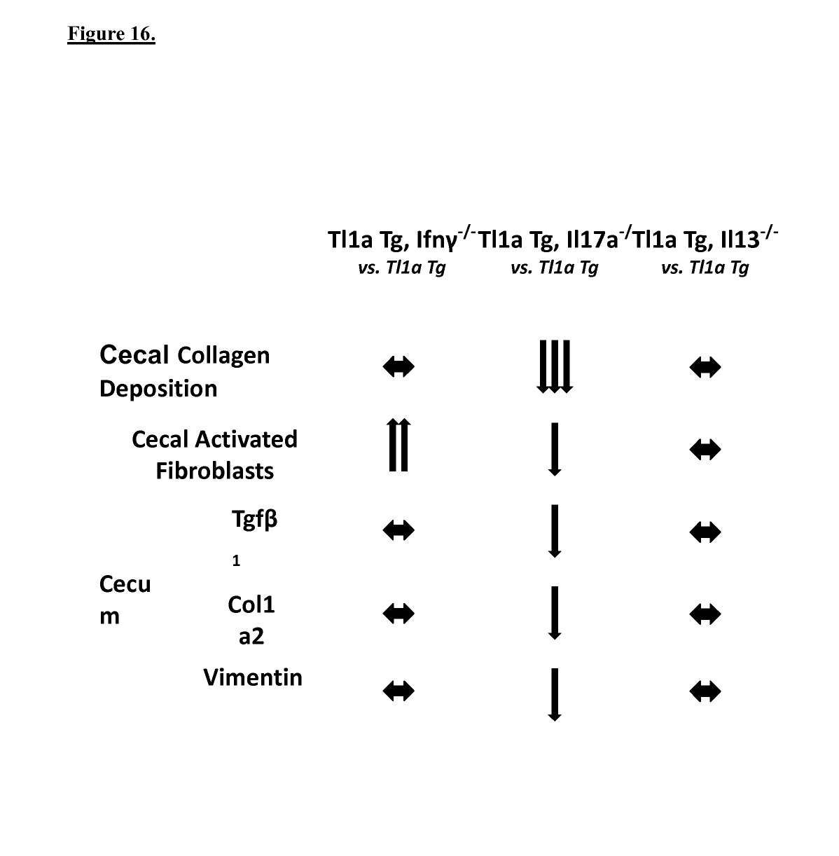

### SUMMARY OF THE INVENTION

Various embodiments herein include a method of treating an inflammatory bowel disease (IBD) related condition in a subject, comprising providing a composition comprising an inhibitor of IL17 signaling, and administering a therapeutically effective dosage of the composition to the subject. In another embodiment, the inhibitor of the IL17 is an IL17 antibody. In another embodiment, the method further comprises administering an inhibitor of TL1A. In another embodiment, the inhibitor of TL1A is a TL1A antibody. In another embodiment, the IBD related condition is fibrosis. In another embodiment, the IBD related condition is a severe form of colitis. In another embodiment, the IBD related condition is inflammation. In another embodiment, the inhibitor of IL17 signaling is an inhibitor of IL17a.

Other embodiments include a method of treating inflammatory bowel disease (IBD) and/or fibrosis in a subject, comprising diagnosing the IBD and/or fibrosis in the subject by determining the level of IFN gamma, IL-17 and/or TL1A expression, and treating the subject. In another embodiment, diagnosing the IBD and/or fibrosis in the subject comprises determining the level of IFN gamma, IL-17 and TL1A expression. In another embodiment, treating the subject comprises administrating a therapeutically effective dosage of a TL1A inhibitor. In another embodiment, the subject is treated by administering a therapeutically effective dosage of TL1A antibody. In another embodiment, treating the subject comprises administering a therapeutically effective dosage of a composition capable of modulating IL-17 activity. In another embodiment, the composition capable of modulating IL-17 activity is an antibody. In another embodiment, treating the subject comprises administering a therapeutically effective dosage of a composition capable of modulating IFN gamma activity. In another embodiment, the subject is treated by surgical procedures. In another embodiment, the method further comprises classifying the diagnosis to select a treatment for the subject. In another embodiment, the method further comprises determining the level of IL13 and/or IL10 expression. In another embodiment, the IL17 is IL17a and/or IL17f.

Other embodiments include a method of treating an inflammatory condition in a subject, comprising diagnosing the inflammatory condition based on the presence or absence of TL1A expression and one or more cytokines, and treating the subject. In another embodiment, the one or cytokines are selected from the group consisting of IFN gamma, IL-17, TL1A, IL13 and/or IL10, In another embodiment, the inflammatory condition comprises gross colonic inflammation, rectal inflammation or cecal inflammation.

Various embodiments include a method of diagnosing an inflammatory bowel disease (IBD) and/or fibrosis subtype in a subject, comprising obtaining a sample from the subject, subjecting the sample to an assay adapted to determining the level of LEN gamma, IL-17 and/or TL1A expression, and diagnosing the subtype, wherein an elevated level of IFN gamma and the presence of TL1A expression is indicative of a severe colitis, and wherein the reduced level of IL-17 is indicative of a less severe form of colitis, inflammation and/or fibrosis. In another embodiment, the assay is quantitative real-time PCR (qRT-PCR). In another embodiment, the assay is an immunoassay. In another embodiment, diagnosing the IBD and/or fibrosis in the subject comprises determining the level of IFN gamma, IL-17 and TL1A expression. In another embodiment, the method further comprises determining the level of IL13 and/or IL10 expression. In another embodiment, the IL17 is IL17a and/or IL17f.

Other embodiments include a method of prognosing inflammatory bowel disease (IBD) and/or fibrosis in a subject, comprising obtaining a sample from the subject, subjecting the sample to an assay adapted to determining the level of IFN gamma, IL-17 and/or TL1A expression, and prognosing the IBD and/or fibrosis in the subject, wherein elevated level of IFN gamma and//or TL1A expression is indicative of a severe colitis, and wherein the reduced level of IL-17 is indicative of a less severe form of colitis, inflammation and/or fibrosis. In another embodiment, the assay is quantitative real-time PCR (qRT-PCR). In another embodiment, the assay is an immunoassay. In another embodiment, diagnosing the IBD and/or fibrosis in the subject comprises determining the level of IFN gamma, IL-17 and TL1A expression. In another embodiment, the method further comprises determining the level of IL13 and/or IL10 expression. In another embodiment, the IL17 is IL17a and/or IL17f.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor NY, 2013); Köhler and Milstein, Derivation of specific antibody-producing tissue culture and tumor lines by cell fusion, Eur, J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

As disclosed herein, the inventors examined the effect of T-helper pathway on TL1A induced colitis, and the effect of T-helper pathway on TL1A induced gut fibrosis. A role for TL1A in gut mucosal inflammation is highlighted by the finding that neutralizing TL1A antibody prevented and treated chronic colitis in mice. However, the contribution of either lymphoid or myeloid derived TL1A to the development of gut inflammation is not fully known. TINA is the product of the TNFSF15 gene that is expressed by both lymphoid and myeloid derived cells. Variants in the TNFSF15 gene have been found to be associated with IBD. The protein product of TNFSF15, TL1A, is elevated in the intestinal mucosa of IBD patients. Certain TNFSF15 haplotypes are associated with susceptibility in non-Jewish Caucasian CD and UC. In addition, TNFSF15 haplotype B is not only associated with risk, but also with severity in Jewish CD patients. Moreover, monocytes from Jewish patients carrying the risk haplotype B express higher levels of TL1A in response to FcγR stimulation. These results show that CD associated TNFSF15 genetic variations contribute to enhanced induction of TL1A, resulting in severe, chronic mucosal inflammation and that modulation of TL1A may be a potential target for therapeutic development. TL1A signals via death domain receptor 3 (DR3) and several studies implicate the TL1A/DR3 signaling pathway in mucosal inflammation. Neutralizing TL1A-antibody ameliorates inflammation in DSS and Gαi2−/− T cell transfer chronic colitis models. Constitutive TL1A expression in mice leads to mild spontaneous ileitis and increased collagen deposition. TL1A modulates the adaptive immune response in the T-helper (Th)-1 effector arm, as shown by TI-1A enhanced interferon (IFN)-γ production from peripheral and mucosal TL1A is a TNF superfamily member Thus, in summary TNFSF15 SNPs are associated with IBD, TNFSF15 haplotype B has increased TL1A expression with a higher risk of small bowel surgery, and constitutive T11a expression in mice confers worsened murine ileo-cecal inflammation and intestinal fibrostenosis. While it is known TL1A can enhance Th1, Th2, and Th17 effector cell function, it is poorly understood which TL1A activated T-helper effector pathway induces intestinal inflammation and fibrosis. Thus, a critical scientific question is understanding the effect of T-helper pathway on TL1A induced colitis and effect of T-helper pathway on TL1A induced gut fibrosis.

Using TL1A-Tg mice crossed to IFN gamma, and to IL-17 knockout mice, the Inventors found that the development of colitis, inflammation and fibrosis, in the presence of constitutive expression of TL1A is heavily dependent upon the presence or absence of particular cytokines. Using adoptive transfer chronic colitis model where the Inventors injected naïve T cells from WT mice, from TL1A Tg mice with sustained TL1A signaling, and from TL1A Tg mice with deficiencies in IFNg, I113, and I117 into RAG KO mice. Mice were sacrificed at 6^{th} weeks post-transfer and analyzed for histologic inflammation, flow cytometry, ELISA, and RT-PCR.

Specifically, in the absence of IFN gamma, TL1A expression results in increased severity of colitis. Alternatively, in the absence of IL-17, TL1A expression does not result in as severe colitis, inflammation and fibrosis, as TL1A overexpression alone. In other words, TL1A driven regional intestinal inflammation and fibrosis is differentially modulated by IFN gamma and IL-17a, and cytokine-cytokine interaction plays an important role to determine severe IBD phenotype and to stratify patients for targeted therapy.

Described herein is a method of treating inflammatory bowel disease (IBD) and/or fibrosis in a subject, including diagnosing the IBD and/or fibrosis in the subject by determining the level of IFN gamma, IL-17 and/or TL1A expression; and treating the subject. In other embodiments, diagnosing the IBD and/or fibrosis in the subject includes determining the level of IFN gamma, IL-17 and TL1A expression. In other embodiments, treating the subject includes administrating a therapeutically effective dosage of a TL1A inhibitor. In other embodiments, the subject is treated by administering a therapeutically effective dosage of TL1A antibody. In other embodiments, treating the subject includes administering a therapeutically effective dosage of a composition capable of modulating IL-17 activity. In other embodiments, the composition capable of modulating IL-17 activity is an antibody. In other embodiments, treating the subject includes administering a therapeutically effective dosage of a composition capable of modulating IFN gamma activity. In other embodiments, the subject is treated by surgical procedures. In other embodiments, the method includes classifying the diagnosis to select a treatment for the subject. In other embodiments, the method includes determining the level of IL13 and/or IL10 expression. In other embodiments, the IL17 is IL17a and/or IL17f. In various embodiments, determining the level of expression can include an absolute measurement or a relative measurement compared to one or more healthy population of individuals, or a relative measurement compared to a clinically relevant population of individuals possessing one or more of the same or different diseases and/conditions as the subject.

Also described herein is a method of treating an inflammatory condition in a subject, including diagnosing the inflammatory condition based on the presence or absence of TL1A expression and one or more cytokines, and treating the subject. In other embodiments, the one or cytokines are selected from the group consisting of: IFN gamma, IL-17, TL1A, IL13 and/or IL10. In other embodiments, the inflammatory condition includes gross colonic inflammation, rectal inflammation or cecal inflammation.

Further described herein is a method of diagnosing an inflammatory bowel disease (MD) and/or fibrosis subtype in a subject, including obtaining a sample from the subject, subjecting the sample to an assay adapted to determining the level of IFN gamma, IL17 and/or TL1A expression, and diagnosing the subtype, wherein an elevated level of IFN gamma and the presence of TL1A expression is indicative of a severe colitis, and wherein the reduced level of IL-17 is indicative of a less severe form of colitis, inflammation and/or fibrosis. In other embodiments, the assay is quantitative real-time PCR (qRT-PCR), In other embodiments, the assay is an immunoassay. In other embodiments, diagnosing the IBD and/or fibrosis in the subject includes determining the level of IFN gamma, IL-17 and TL1A expression. In other embodiments, the method further includes determining the level of IL13 and/or IL10 expression. In other embodiments, the IL17 is IL17a and/or IL17f. In various embodiments, determining the level of expression can include an absolute measurement or a relative measurement compared to one or more healthy population of individuals, or a relative measurement compared to a clinically relevant population of individuals possessing one or more of the same or different diseases and/conditions as the subject.

Also described herein is a method of prognosing inflammatory bowel disease (IBD) and/or fibrosis in a subject, including obtaining a sample from the subject, subjecting the sample to an assay adapted to determining the level of IFN gamma, IL-17 and/or TL1A expression, and prognosing the IBD and/or fibrosis in the subject, wherein elevated level of IFN gamma and/or TL1A expression is indicative of a severe colitis, and wherein the reduced level of IL-17 is indicative of a less severe form of colitis, inflammation and/or fibrosis. In other embodiments, the assay is quantitative real-time PCR (qRT-PCR). In other embodiments, the assay is an immunoassay. In other embodiments, diagnosing the IBD and/or fibrosis in the subject includes determining the level of IFN gamma, IL-17 and TL1A expression. In other embodiments, the method includes determining the level of IL13 and/or IL10 expression. In other embodiments, the IL17 is IL17a and/or IL17f. In various embodiments, determining the level of expression can include an absolute measurement or a relative measurement compared to one or more healthy population of individuals, or a relative measurement compared to a clinically relevant population of individuals possessing one or more of the same or different diseases and/conditions as the subject.

Described herein is a method of diagnosing an IBD and/or fibrosis subtype in a subject, including obtaining a sample from the subject, subjecting the sample to an assay adapted to determine the presence or absence of IFN gamma, IL-17 and/or TL1A related biomarkers, diagnosing the subtype, wherein the absence of IFN gamma and the presence of TL1A related biomarkers is indicative of a severe colitis, and wherein the absence of IL-17 is indicative of a less severe form of colitis, inflammation and/or fibrosis. In other embodiments, the method includes determining determine the presence or absence of IL-17 is and/or IL10 related biomarkers. In other embodiments, the IL17 is IL17a and/or IL17f.

Further described herein is a method of prognosing IBD and/or fibrosis in a subject, including obtaining a sample from the subject; subjecting the sample to an assay adapted to determine the presence or absence of IFN gamma, IL-17 and/or TL1A related biomarkers, and prognosing the IBD and/or fibrosis in the subject, wherein the absence of IFN gamma and the presence of TL1A related biomarker expression is indicative of a severe colitis, and wherein the absence of IL-17 is indicative of a less severe form of colitis, inflammation and/or fibrosis. In other embodiments, the method includes determining determine the presence or absence of IL13 and/or IL10 related biomarkers. In other embodiments, the IL17 is IL17a and/or IL17f.

Also described herein is a method of treating an inflammatory condition in a subject, including diagnosing the inflammatory condition based on the presence or absence of TL1A related biomarker expression and one or more cytokines, and treating the subject.

Further described herein is a method of treating IBD and/or fibrosis in a subject, including diagnosing the IBD and/or fibrosis in the subject by determining the presence or absence of IFN gamma, IL-17 and/or TL1A related biomarker expression, and treating the subject. In some embodiments, treating the subject includes administrating a therapeutically effective dosage of a TL1A inhibitor. In some embodiments, the subject is treated by administering a therapeutically effective dosage of TL1A antibody. In some embodiments, the subject is treated by surgical procedures, in other embodiments, the method includes determining determine the presence or absence of IL13 and/or IL10 related biomarkers. In other embodiments, the IL17 is IL17a and/or IL17f.

In one embodiment, the present invention is a method of diagnosing a condition in a subject, by obtaining a sample from a subject, assaying the sample to determine the presence or absence of a IFN gamma and/or IL-17 related biomarker, and diagnosing the subject.

In another embodiment, the present invention provides a method of diagnosing an IBD and/or fibrosis subtype in a subject, comprising obtaining sample from the subject, assaying the sample to determine the presence or absence of a IFN gamma, IL-17, and/or TL1A related biomarker, and diagnosing the IBD and/or fibrosis subtype. In another embodiment, the absence of IFN gamma and the presence of TL1A expression is indicative of a severe colitis. In another embodiment, the absence of IL17 is indicative of a less severe form of colitis, inflammation and/or fibrosis. In other embodiments, the method includes determining determine the presence or absence of IL13 and/or IL10 related biomarkers. In other embodiments, the IL17 is IL17a and/or IL17f.

In another embodiment, the present invention provides a method of prognosing IBD and/or fibrosis in a subject, comprising obtaining sample from the subject, assaying the sample to determine the presence or absence of IFN gamma, IL-17, and/or TL1A related biomarkers, and prognosing the condition wherein the absence of IFN gamma and the presence of TL1A related biomarkers is indicative of a severe colitis, and the absence of IL-17 related biomarkers is indicative of a less severe form of colitis, inflammation and/or fibrosis. In other embodiments, the method includes determining determine the presence or absence of IL13 and/or IL10 related biomarkers. In other embodiments, the IL17 is IL71a and/or IL17f.

In one embodiment, the present invention provides a method of treating a disease in a subject, comprising obtaining sample from the subject, assaying the sample to determine the presence or absence of IFN gamma, IL-17, and/or TL1A related biomarkers, and treating the subject. In another embodiment, the disease is IBD and/or fibrosis subtype. In another embodiment, the absence of IFN gamma and the presence of TL1A related biomarkers is indicative of a severe colitis. In another embodiment, the absence of IL-17 related biomarkers is indicative of a less severe form of colitis, inflammation and/or fibrosis. In another embodiment, the subject is treated by administrating a therapeutically effective dosage of TL1A inhibitor. In another embodiment, the subject is treated by administering a therapeutically effective dosage of TL1A antibody. In another embodiment, the subject is treated by surgical procedures. In other embodiments, the method includes determining determine the presence or absence of IL13 and/or IL10 related biomarkers. In other embodiments, the IL17 is IL17a and/or IL17f.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Overview

The inventors examined the effect of T-helper pathway on TL1A induced colitis, and the effect of T-helper pathway on TL1A induced gut fibrosis. Using TL1A-Tg mice crossed to IFN gamma, and to IL-17 knockout mice, the inventors found that the development of colitis, inflammation and fibrosis, in the presence of constitutive expression of TL1A is heavily dependent upon the presence or absence of particular cytokines. Specifically, in the absence of IFN gamma, TL1A expression results in increased severity of colitis. Alternatively, in the absence of IL-17, TL1A expression does not result in as severe colitis, inflammation and fibrosis, as TL1A overexpression alone. In other words, TL1A driven regional intestinal inflammation and fibrosis is differentially modulated by IFN gamma and IL-17a, and cytokine-cytokine interaction plays an important role to determine severe IBD phenotype and to stratify patients for targeted therapy.

### Example 2

### Gross Colonic Inflammation

Gross Colonic inflammation is represented by increased erythema and swelling. WT mice in the adoptive transfer model have increased inflammation in the rectum, in contrast to WT, the inflammation was shifted to the cecum under T11a driven condition.

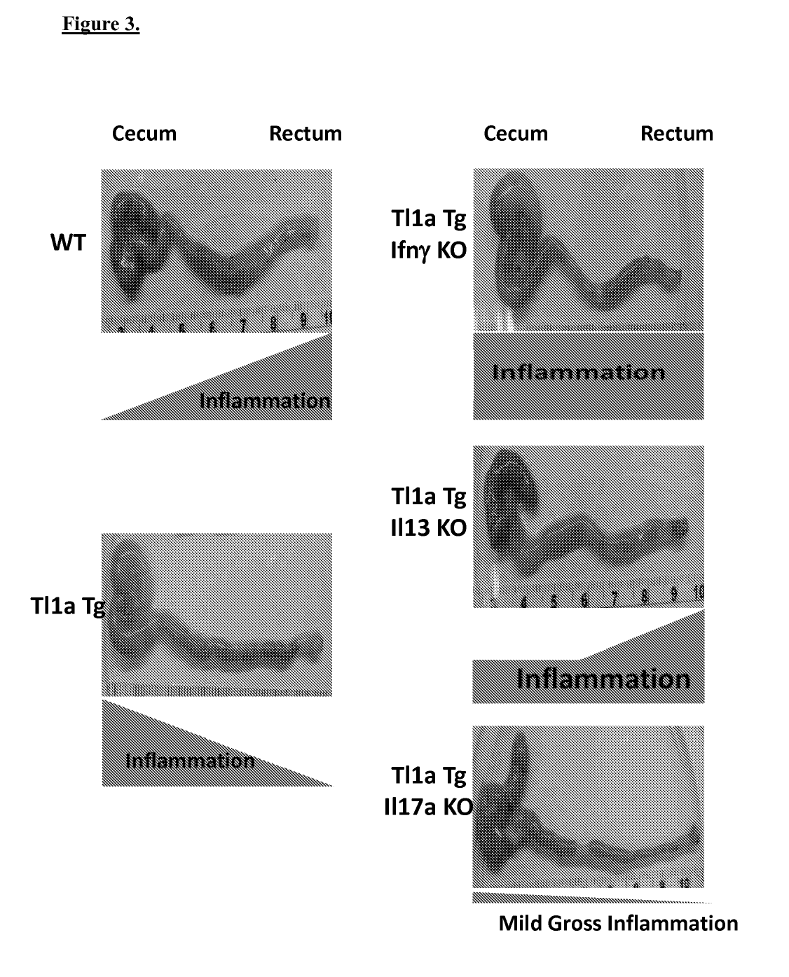

Combining effector cytokine deficiency with sustained TL1A expression modulated regional gross inflammation, under TL1a driven condition, IFNg deficiency led to pan colitis, IL13 deficiency shifted the inflammation to the WT pattern, and IL17 deficiency reduced overall colonic inflammation as shown in FIG. 3.

### Example 3

### IL17a KO Reduced TL1A Associated Proximal Colitis

Mice with sustained TL1A expression have worsened cecal inflammation compared to WT. To see whether Th effector immune pathway modulated cecal inflammation under TL1A driven condition, the Inventors quantitated the degree of cecal inflammation in mice with sustained TL1A expression in the setting of IL13, IFNg, and IL17a deficiency.

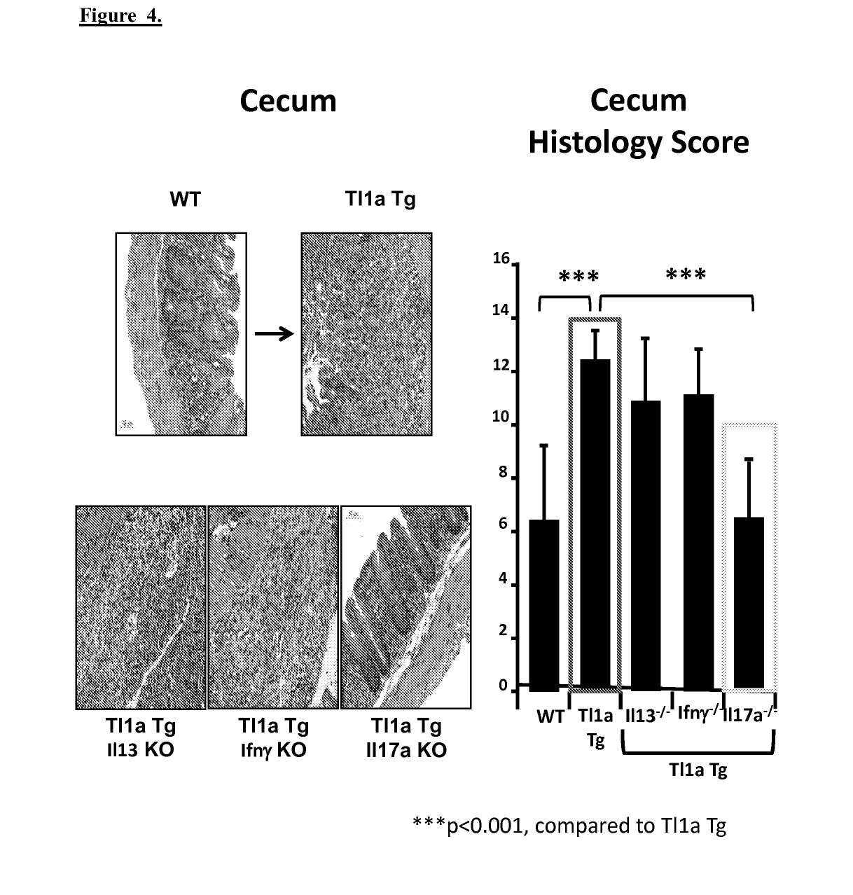

Compared to TL1A tg alone, there is no differences in inflammation with IL13 and IFNg deficiency. Shown here are results indicating that IL17a deficiency significantly reduced the severity of TL1A associated cecal inflammation (FIG. 4). Thus, it is shown that IL17a KO reduces TL1A associated proximal colitis.

### Example 4

### Rectal Sparing of Inflammation is Modulated by Th Effector Response

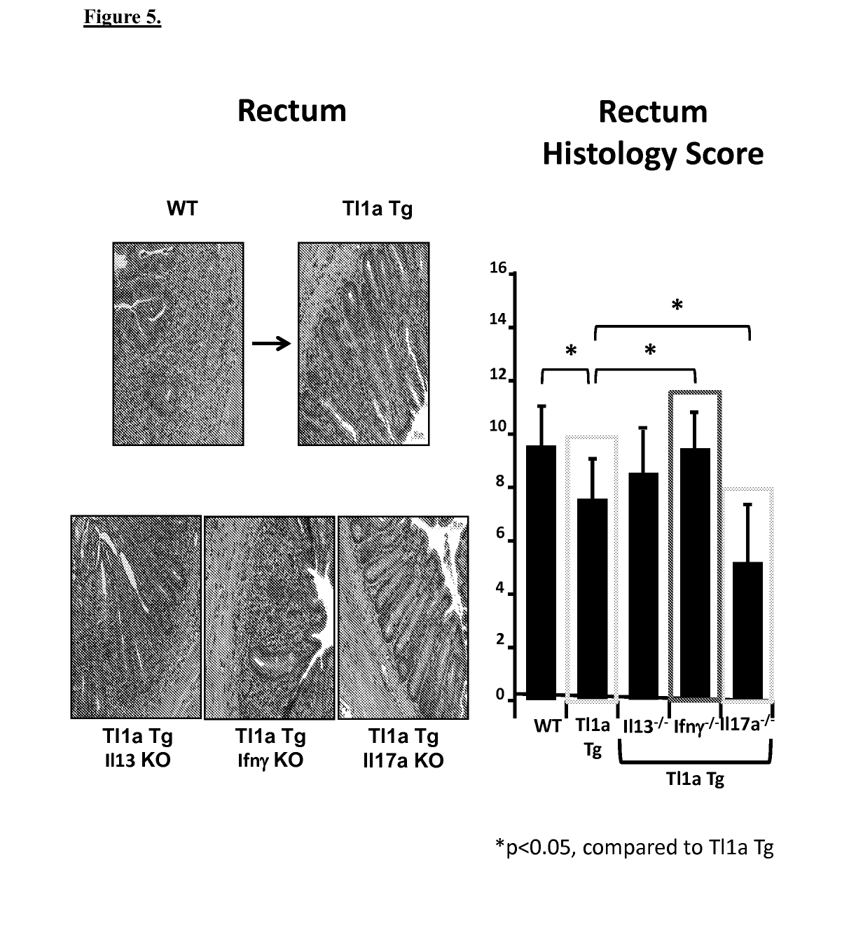

Previous studies show that mice with sustained TL1A expression have rectal sparing of inflammation under colitogenic condition. To see whether Th effector immune pathway modulated sparing of rectal inflammation under TL1A driven condition, the Inventors quantitated the degree of rectal inflammation in mice with sustained TL1A expression in the setting of IL13, IFNg and IL17a deficiency. Shown here are results indicating that IFNg deficiency significantly abrogated the severity of TL1A associated sparing of rectal inflammation (FIG. 5). Additionally, IL17a deficiency further improved the rectal sparing associated with sustained TL1A expression, thereby demonstrating rectal sparing of inflammation is modulated by Th effector response.

### Example 5

### IFN Gamma is Reduced with IL17a Deficiency Under TL1A Driven Condition

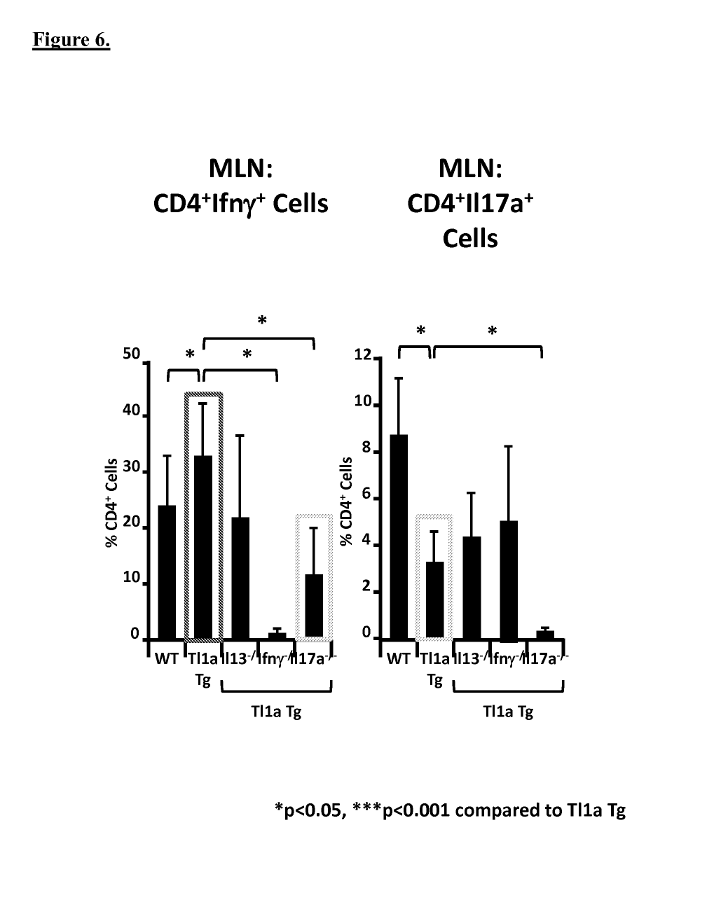

To assess the modulation of regional colonic inflammation by effector Th response under TL1A driven condition, the Inventors performed flow cytometry analysis. Consistant with previous findings, sustained expression of TL1A led to increased percentage of CD4+IFNg+ cells and decreased percentage of CD4+I117a+ cells as compared to WT. Under TL1A driven condition, IL17a deficiency reduced CD430 IFNg+ cells (FIG. 6), thereby demonstrating IFN gamma is reduced with IL17a deficiency under TL1A driven condition.

### Example 6

### Sustained TL1A Expression with IFN Gamma and IL17a Deficiency Increased IL17f

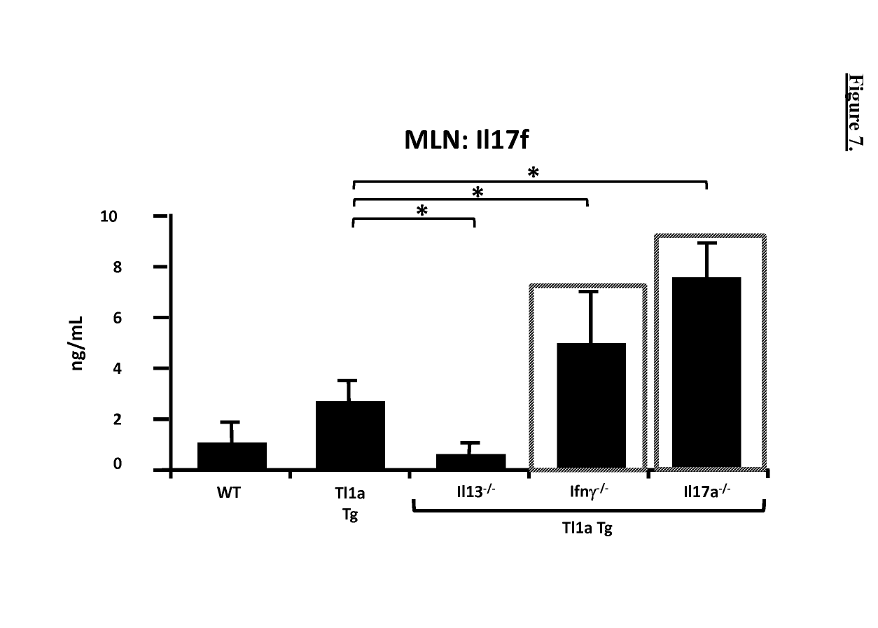

The Inventors isolated cells from the MLN and measured Th17 related cytokines production by ELISA. Consistent with our previous finding, it was discovered that IL17a production was increased in mice with sustained TL1A expression. However, IFNg and I113 deficiency under TL1A driven condition didn't alter I117a production when compared to TL1A tg mice alone. Another major IL17 cytokine is IL17F. The results further suggest that IL17f production was increased in RAG mice that received T11a Tg, naïve T cells with deficiencies in IFNg and IL17 KO (FIG. 7), thereby demonstrating that sustained TL1A expression with IFN gamma and IL17a deficiency increased IL17f.

### Example 7

### IFN Gamma Deficiencies Modulate TL1A Driven TH-2 Responses

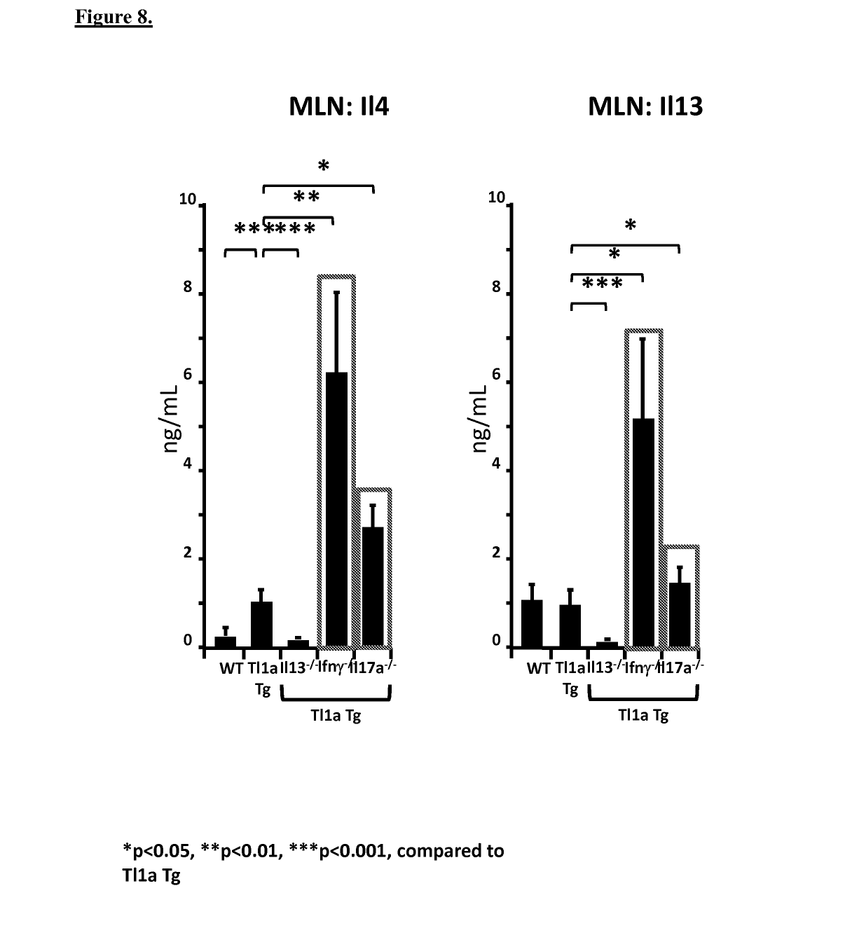

Th2 associated cytokines production were also measured. We found that I14 and I113 were increased in both IFNg deficiency and I117a deficiency under TL1A driven condition. The enhancement in Th2 related cytokine is higher with IFNg KO than IL17a KO (FIG. 8), thereby demonstrating that IFN gamma deficiencies modulate TL1A driven TH-2 responses.

### Example 8

### Increased IL10 Under TL1A Driven Condition with IL17a Deficiency

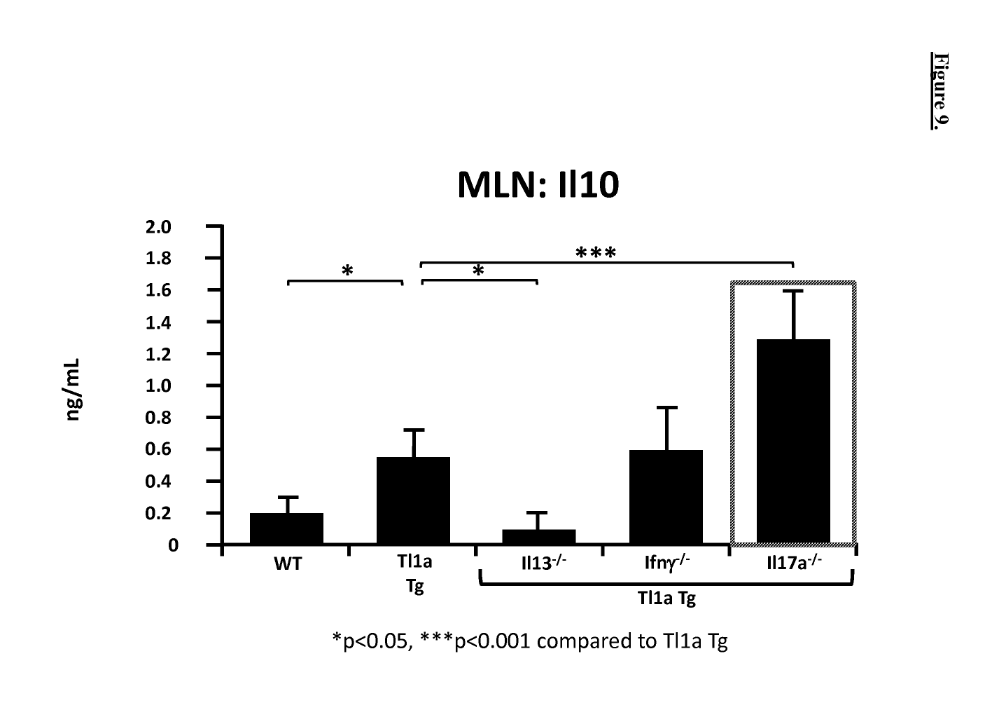

IL10, a regulatory cytokine, was also measured and found to be significantly increased in RAG mice that received TH a Tg, IL17 KO naïve T cells (FIG. 9), demonstrating increased IL10 under TL1A driven condition with IL17a deficiency.

### Example 9

### I117a Deficiency Reduces TL1A Mediated Gut Fibrosis

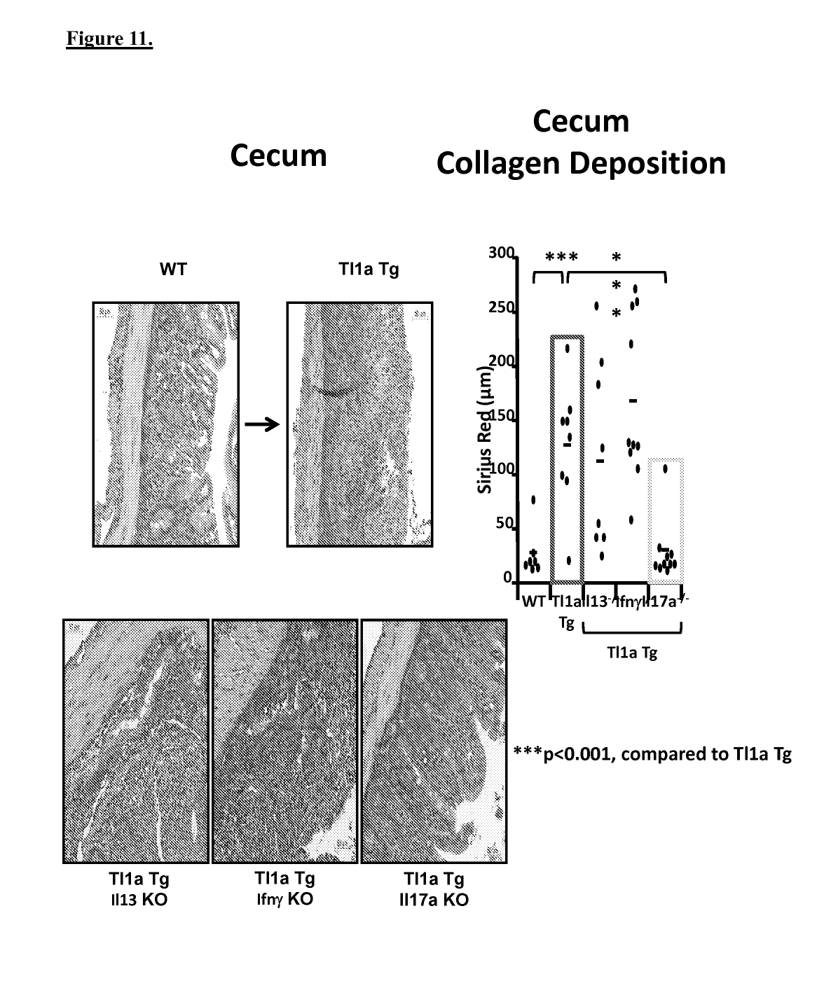

TL1A can enhance gut fibrosis as shown here with increased sirius red stain that stains collagen red in the TL1A Tg mice as compared to WT mice. Under T11a driven condition, blocking IFNg has no effect on fibrosis whereas blocking IL17 significantly reduces collagen deposition when compared to TL1A transgenic mice (FIG. 11) thereby demonstrating IL17a deficiency reduces TL1A mediated gut fibrosis.

### Example 10

### Activated Fibroblasts are Increased by IFN Gamma KO but Reduced by IL17a KO

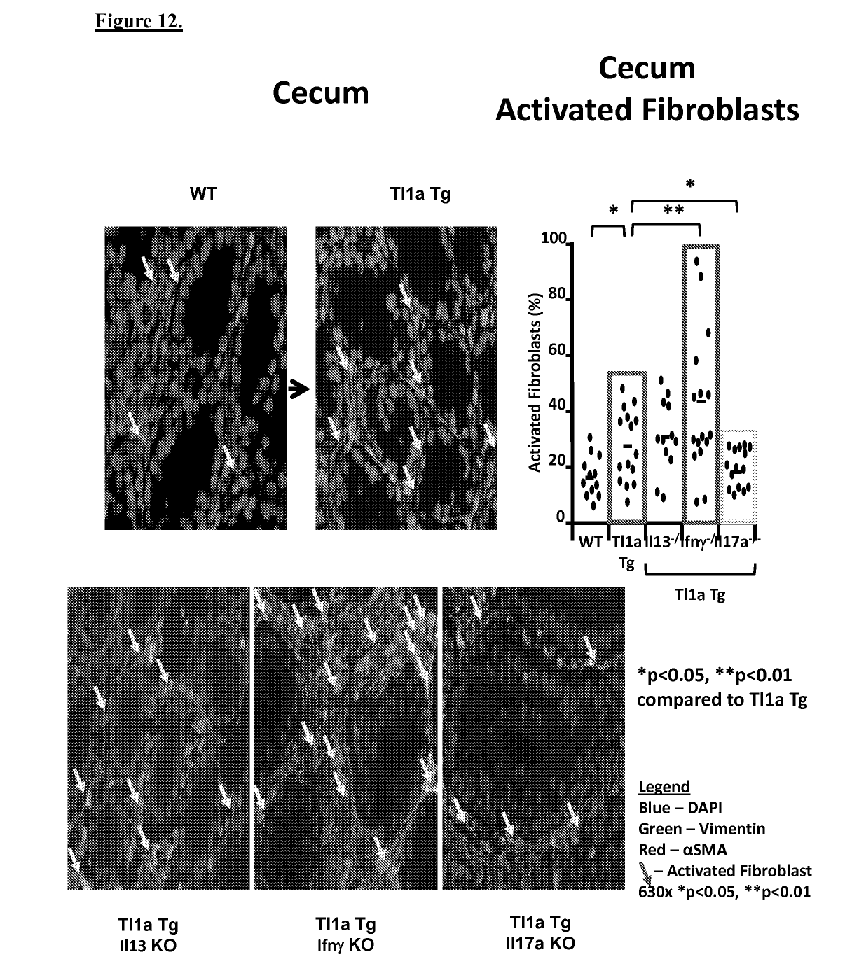

To assess whether the different degree of collagen deposition is due to the different numbers of activated myofibroblasts, the Inventors performed immunofluorescent staining for vimentin in green that stains all fibroblasts and alpha SMA in red that stains activated myofibroblasts. Under TL1A driven condition, activated myofibroblasts were increased compared to WT, blocking IFNg further increased activated myofibroblasts whereas blocking IL17 reduced activated myofibroblasts when compared to TL1A transgenic mice (FIG. 12), thereby demonstrating activated fibroblasts are increased by IFN gamma KO but reduced by IL17a KO.

### Example 11

### IL17a Modulates Fibrogenic Factors Expression

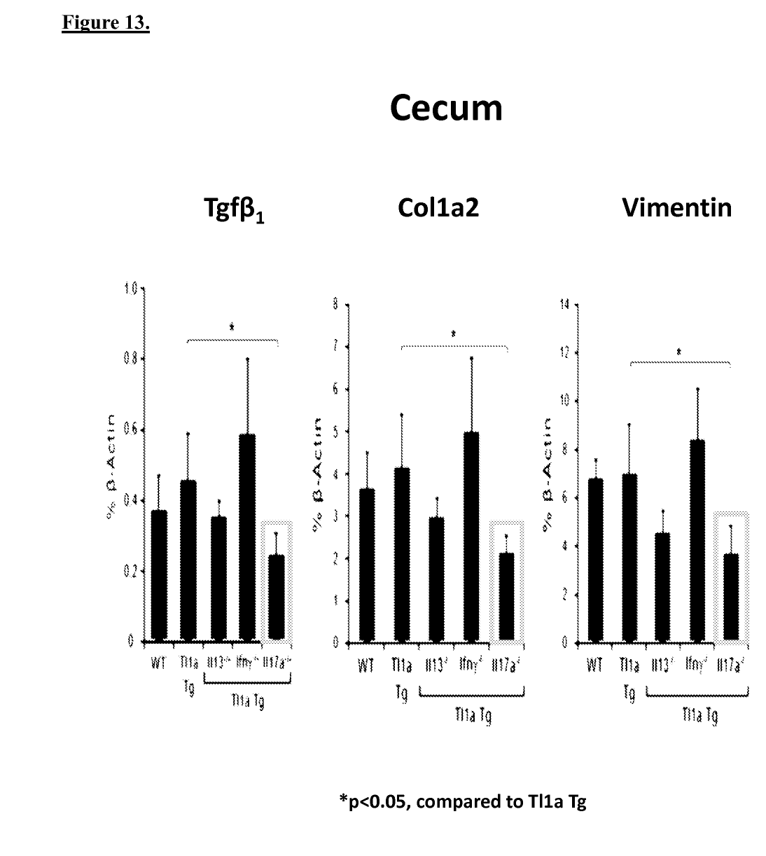

For different fibrosis patterns, it was discovered that under T11a driven condition, I117a deficiency reduced expression of pro-fibrotic factors including TGFb1, and also reduced expressions of fibrotic mediators Col1a2 and vimentin, which is consistent with reduced intestinal fibrosis in these mice. (FIG. 13) thereby demonstrates that under TL1A driven conditions, IL17a modulates fibrogenic factors expression.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the methods of prognosis and diagnosis for inflammatory bowel disease related diseases and/or conditions, compositions of generated by the aforementioned techniques, treatment of diseases and/or conditions that relate to the teachings of the invention, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the Wowing claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of treating an inflammatory bowel disease (IBD) related condition in a subject, comprising:
providing a composition comprising an inhibitor of IL17 signaling; and
administering a therapeutically effective dosage of the composition to the subject.

2. The method of claim 1, wherein the inhibitor of the IL17 is an IL17 antibody.

3. The method of claim 1, further comprising administering an inhibitor of TL1A.

4. The method of claim 3, wherein the inhibitor of TL1A is a TL1A antibody.

5. The method of claim 1, wherein the IBD related condition is fibrosis, a severe form of colitis or inflammation.

6. (canceled)

7. (canceled)

8. The method of claim 1, wherein the inhibitor of IL17 signaling is an inhibitor of IL17a.

9. A method of treating inflammatory bowel disease (IBD) and/or fibrosis in a subject, comprising:
diagnosing the IBD and/or fibrosis in the subject by determining the level of IFN gamma, IL-17 and/or TL1A expression; and
treating the subject.

10. The method of claim 9, wherein diagnosing the IBD and/or fibrosis in the subject comprises determining the level of IFN gamma, IL-17 and TL1A expression.

11. The method of claim 9, wherein treating the subject comprises administrating a therapeutically effective dosage of a TL1A inhibitor, administering a therapeutically effective dosage of a composition capable of modulating IL-17 activity, administering a therapeutically effective dosage of a composition capable of modulating IFN gamma activity.

12. The method of claim 9, wherein the subject is treated by administering a therapeutically effective dosage of TL1A antibody.

13. (canceled)

14. The method of claim 11, wherein the composition capable of modulating IL-17 activity is an antibody.

15. (canceled)

16. The method of claim 9, wherein the subject is treated by surgical procedures.

17. The method of claim 9, further comprising classifying the diagnosis to select a treatment for the subject.

18. The method of claim 9, further comprising determining the level of IL13 and/or IL10 expression.

19. The method of claim 9, wherein the IL17 is IL17a and/or IL17f.

20. A method of treating an inflammatory condition in a subject, comprising:
diagnosing the inflammatory condition based on the presence or absence of TL1A expression and one or more cytokines; and
treating the subject.

21. The method of claim 20, wherein the one or cytokines are selected from the group consisting of: IFN gamma, IL-17, TL1A, IL13 and/or IL10.

22. The method of claim 20, wherein the inflammatory condition comprises gross colonic inflammation, rectal inflammation or cecal inflammation.

23. A method of diagnosing an inflammatory bowel disease (IBD) and/or fibrosis subtype in a subject, comprising:
obtaining a sample from the subject;
subjecting the sample to an assay adapted to determining the level of IFN gamma, IL-17 and/or TL1A expression; and
diagnosing the subtype,
wherein an elevated level of IFN gamma and the presence of TL1A expression is indicative of a severe colitis, and
wherein the reduced level of IL-17 is indicative of a less severe form of colitis, inflammation and/or fibrosis.

24. The method of claim 23, wherein the assay is quantitative real-time PCR (qRT-PCR) or an immunoassay.

25. (canceled)

26. The method of claim 23, wherein diagnosing the IBD and/or fibrosis in the subject comprises determining the level of IFN gamma, IL-17 and TL1A expression.

27. The method of claim 23, further comprising determining the level of IL13 and/or IL10 expression.

28. The method of claim 23, wherein the IL17 is IL17a and/or IL17f.

29. A method of prognosing inflammatory bowel disease (IBD) and/or fibrosis in a subject, comprising:
obtaining a sample from the subject;
subjecting the sample to an assay adapted to determining the level of IFN gamma, IL-17 and/or TL1A expression; and
prognosing the IBD and/or fibrosis in the subject,
wherein elevated level of IFN gamma and//or TL1A expression is indicative of a severe colitis, and
wherein the reduced level of IL-17 is indicative of a less severe form of colitis, inflammation and/or fibrosis.

30. The method of claim 29, wherein the assay is quantitative real-time PCR (qRT-PCR) or an immunoassay.

31. (canceled)

32. The method of claim 29, wherein diagnosing the IBD and/or fibrosis in the subject comprises determining the level of IFN gamma, IL-17 and TL1A expression.

33. The method of claim 29, further comprising determining the level of IL13 and/or IL10 expression.

34. The method of claim 29, wherein the IL17 is IL17a and/or IL17f.
